(19) United States
(12) Patent Application Publication
(10) Pub. No.: US 2026/0151591 A1
(43) Pub. Date: Jun. 4, 2026

(54) CATHETER SYSTEM HAVING A VENT PLUG AND RELATED METHODS

(71) Applicant: Becton, Dickinson and Company, Franklin Lakes, NJ (US)

(72) Inventors: Jonathan Karl Burkholz, Salt Lake City, UT (US); Mukilan Balamurugan, Tamil Nadu (IN); Edwin Jeyaseelan A., Tamil Nadu (IN); Praveen Nalawade, Karnataka (IN)

(21) Appl. No.: 18/965,819

(22) Filed: Dec. 2, 2024

(51) Int. Cl. A61M 25/00; A61M 25/06; A61M 39/10

(52) U.S. Cl. CPC A61M 25/0043; A61M 25/0693; A61M 39/10

(57) ABSTRACT

A catheter system may include a catheter adapter and a catheter extending distally from a distal end of the catheter adapter. The catheter system may include an extension tube in fluid communication with the catheter adapter and the catheter. The catheter system may include a connector in fluid communication with the extension tube. The connector may include a septum disposed within a body. The catheter system may include a vent plug movable from a first position and to a second position. A venting pathway of the vent plug may be permeable to air and not blood. When the vent plug is in the first position, the septum may seal the body. In response to movement of the vent plug from the first position to the second position, the vent plug may be configured to penetrate the septum such that air is vented through the vent plug.

### BACKGROUND

Catheters are commonly used for a variety of infusion therapies. For example, catheters may be used for infusing fluids, such as normal saline solution, various medicaments, and total parenteral nutrition, into a patient. Catheters may also be used for withdrawing blood from the patient.

Referring to FIGS. 1A-B, a prior art catheter system 10 having a needle assembly 12 configured for insertion of an “over the needle” catheter 14 is depicted. The prior art catheter system 10 generally includes the catheter 14 having a distal end 16 for insertion into a vein, a proximal end 18, and a flexible wall defining a lumen extending between the distal end 16 and the proximal end 18. Frequently, the proximal end 18 of the catheter 14 is coupled to a catheter adapter 20. The catheter 14 can be coupled to the needle assembly 12 by positioning the catheter 14 coaxially over an introducer needle 22 of the needle assembly 12. The catheter 14 thus rides with the introducer needle 22 through the skin, tissue, and vein wall and into a vein of a patient. Once the catheter 14 has been entered into the vein, the catheter 14 can be advanced further into the vein as desired, and the introducer needle 22 can be withdrawn from the catheter 14. The catheter 14 can then be secured into place on the patient and connected to an IV fluid supply or blood collection device, for example.

In some instances, the prior art catheter system 10 can include an extension tube 24 having a clamp 26 and an adapter 28 for connection to the IV fluid supply or the blood collection device. In order to verify proper placement of the introducer needle 22 and the catheter 14 in the vein, a clinician generally confirms that there is flashback of blood visible in one or more of a flashback chamber of the catheter adapter 20, between the introducer needle 22 and the catheter 14, and within the extension tube 24. During insertion of the introducer needle 22 and the catheter 14 into the vein, the clamp 26 is open, and a vent plug 30 is generally coupled directly to the adapter 28 to allow air to escape from the prior art catheter system 10, thereby enabling the blood flashback to flow into one or more of the flashback chamber of the catheter adapter 20, between the introducer needle 22 and the catheter 14, and within the extension tube 24.

After placement of the introducer needle 22 has been confirmed, the clinician closes or clamps the clamp 26 to halt the progression of blood through the prior art catheter system 10, removes the vent plug 30, and replaces the vent plug 30 with another device such as the IV fluid supply or the blood collection device. The clinician then unclamps the clamp 26, flushes the blood from the prior art catheter system 10 back into the vasculature of the patient, and re-clamps the clamp 26. By removing the vent plug 30 from the adapter 28, the fluid path of the prior art catheter system 10 is temporarily exposed to potential contamination from the external environment. Moreover, removing the vent plug 30 from the adapter 28 may lead to blood exposure if blood leaks from the adapter 28.

The subject matter claimed herein is not limited to embodiments that solve any disadvantages or that operate only in environments such as those described above. Rather, this background is only provided to illustrate one example technology area where some implementations described herein may be practiced.

### SUMMARY

In some embodiments, a catheter system may include a catheter adapter, which may include a distal end and a proximal end. In some embodiments, the catheter system may include a catheter extending distally from the distal end of the catheter adapter. In some embodiments, the catheter system may include an extension tube in fluid communication with the catheter adapter and the catheter. In some embodiments, the catheter system may include a connector proximal to the extension tube and in fluid communication with the extension tube. In some embodiments, the connector may include a needleless access connector. In some embodiments, the connector may include a body and a septum disposed within the body.

In some embodiments, the catheter system may include a vent plug movable from a first position to a second position. In some embodiments, the vent plug may include a venting pathway that is permeable to air and not blood. In some embodiments, when the vent plug is in the first position, the septum may seal the body. In some embodiments, in response to movement of the vent plug from the first position to the second position, the vent plug may be configured to penetrate the septum such that air is vented through the septum and the vent plug via the venting pathway.

In some embodiments, in response to the vent plug being in the second position, blood flashback may configured to travel into the extension tube. In some embodiments, the catheter adapter may include a side port disposed between the distal end of the catheter adapter and the proximal end of the catheter adapter. In some embodiments, the extension tube may include a distal end integrated with the side port. In some embodiments, the extension tube may include a proximal end integrated with an adapter having a port. In some embodiments, the connector may be coupled to the port of the adapter. In some embodiments, the adapter may include a Y-adapter or another suitable adapter.

In some embodiments, the connector may include a proximal end having exterior threading. In some embodiments, the vent plug may include interior threading. In some embodiments, the exterior threading may be spaced apart from the interior threading when the vent plug is in the first position. In some embodiments, the vent plug may be configured to be pushed distally from the first position until the interior threading contacts the exterior threading. In some embodiments, after the interior threading contacts the exterior threading, the interior threading may be configured to be threaded to the exterior threading to move the vent plug to the second position.

In some embodiments, the vent plug may include an extension configured to penetrate the septum in response to movement of the vent plug from the first position to the second position. In some embodiments, the extension may include a tapered male luer. In some embodiments, the venting pathway may extend through an interior of the extension. In some embodiments, the vent plug may include a filter membrane disposed at a proximal end of the venting pathway or a distal end of the venting pathway. In some embodiments, the venting pathway may include one or more grooves disposed on an exterior of the extension, and the grooves may be permeable to air and not blood.

In some embodiments, the connector may include an outer surface having an annular groove. In some embodiments, the vent plug may include multiple arms disposed within the annular groove in the first position to resist proximal movement of the vent plug with respect to the connector. In some embodiments, the outer surface of the connector may include multiple grooves. In some embodiments, the multiple arms of the vent plug may be disposed within the grooves. In some embodiments, in response to movement of the vent plug from the first position to the second position, the multiple arms are configured to slide distally within the multiple grooves.

In some embodiments, the catheter system may include a housing coupled to the connector and having an interior protrusion. In some embodiments, the vent plug may include an exterior protrusion. In some embodiments, in response to movement of the vent plug from the first position to the second position, the exterior protrusion may be configured to snap past the interior protrusion to resist return of the vent plug to the first position. In some embodiments, the interior protrusion may be annular, and/or the exterior protrusion may be annular.

In some embodiments, the housing may include another interior protrusion proximal to the interior protrusion. In some embodiments, when the vent plug is in the first position, the other interior protrusion may contact a proximal surface of the exterior protrusion to resist proximal movement of the vent plug with respect to the housing.

In some embodiments, a distal end of the housing may include threading threaded to the connector. In some embodiments, the vent plug may include a stepped surface forming a push tab at a proximal end of the vent plug. In some embodiments, in response to movement of the vent plug from the first position to the second position, the push tab may contact a proximal end of the housing.

In some embodiments, the vent plug may include the extension configured to penetrate the septum in response to movement of the vent plug from the first position to the second position. In some embodiments, the vent plug may include a closed proximal end. In some embodiments, the extension may include a first hole permeable to air and not to blood. In some embodiments, the vent plug may include a second hole, which may be permeable to air and not to blood. In some embodiments, when the vent plug is in the first position, the first hole may be disposed within the septum, sealing the first hole. In some embodiments, in response to movement of the vent plug from the first position to the second position, the first hole may be positioned proximal to the septum, and air is configured to vent through the first hole and the second hole.

In some embodiments, the connector may include the proximal end having the exterior threading, and the vent plug may include a collar having the interior threading. In some embodiments, the interior threading may be unthreaded with respect to the exterior threading to move the vent plug from the first position to the second position. In some embodiments, the collar may include the second hole.

In some embodiments, the vent plug may include particular multiple arms having inwardly extending protrusions. In some embodiments, when the vent plug is disposed in the first position, the inwardly extending protrusions may snap onto the exterior threading of the connector. In some embodiments, the particular multiple arms may be configured to bias outwardly to remove the vent plug from the connector for disposal.

In some embodiments, a method of observing blood flashback to confirm placement of the catheter within a blood vessel may include inserting the catheter system into a patient. In some embodiments, when the vent plug is in the first position, the septum may seal the body such that the catheter system is a closed catheter system. In some embodiments, after inserting the catheter system into the patient, the method may include moving the vent plug from the first position to the second position. In some embodiments, in response to movement of the vent plug from the first position to the second position, the vent plug may penetrate the septum such that air is vented through the septum and the vent plug, and blood flashback is observed in the extension tube. In some embodiments, when the vent plug is in the second position, the closed catheter system may be maintained, thereby preventing blood from the patient from escaping from the catheter system to the ambient environment.

It is to be understood that both the foregoing general description and the following detailed description are examples and explanatory and are not restrictive of the invention, as claimed. It should be understood that the various embodiments are not limited to the arrangements and instrumentality shown in the drawings. It should also be understood that the embodiments may be combined, or that other embodiments may be utilized and that structural changes, unless so claimed, may be made without departing from the scope of the various embodiments of the present invention. The following detailed description is, therefore, not to be taken in a limiting sense.

### BRIEF DESCRIPTION OF THE SEVERAL VIEWS OF THE DRAWINGS

Example embodiments will be described and explained with additional specificity and detail through the use of the accompanying drawings in which: FIG. 1A is an upper perspective view of a prior art catheter system;

FIG. 1B is a cross-sectional view of the prior art catheter system;

FIG. 2A is an upper perspective view of an example catheter system having an example vent plug, according to some embodiments;

FIG. 2B is an upper perspective view of the vent plug coupled to an example connector, illustrating the vent plug in an example first position, according to some embodiments;

FIG. 2C is an upper perspective view of the vent plug coupled to the connector, illustrating the vent plug in an example second position, according to some embodiments;

FIG. 2D is a lower perspective view of the vent plug, according to some embodiments;

FIG. 2E is an upper perspective view of the vent plug, according to some embodiments;

FIG. 2F is a cross-sectional view of the vent plug coupled to the connector, illustrating the vent plug in the first position, according to some embodiments;

FIG. 2G is an upper perspective view of the vent plug coupled to the connector, illustrating the vent plug in the second position, according to some embodiments;

FIG. 2H is a lower perspective view of the vent plug, illustrating example grooves on an outer surface of the vent plug, according to some embodiments;

FIG. 2I is an upper perspective view of the vent plug coupled to the connector, illustrating the vent plug in the first position and having the grooves on the outer surface, according to some embodiments;

FIG. 2J is a cross-sectional view of the vent plug coupled to the connector, illustrating the vent plug in the second position and having the grooves on the outer surface, according to some embodiments;

FIG. 2K is an upper perspective view of the vent plug having example arms extending 120° around a circumference of the vent plug, according to some embodiments;

FIG. 2L is a lower perspective view of the vent plug having the arms extending 120° around the circumference of the vent plug, according to some embodiments;

FIG. 2M is an upper perspective view of the vent plug, illustrating the arms with example grooves, according to some embodiments;

FIG. 2N is an upper perspective view of the vent plug, illustrating the arms having example inwardly extending protrusions, according to some embodiments;

FIG. 2O is an upper perspective view of the vent plug, illustrating the arms having example inwardly extending protrusions, according to some embodiments;

FIG. 2P is an upper perspective view of the vent plug, illustrating the arms having example interior threading, according to some embodiments;

FIG. 2Q is a lower perspective view of the vent plug, illustrating the arms having the interior threading of FIG. 2P, according to some embodiments;

FIG. 2R is a lower perspective view of the vent plug, illustrating an example annular ring, according to some embodiments;

FIG. 3A is an upper perspective view of the catheter system having an example vent plug, according to some embodiments;

FIG. 3B is an upper perspective view of the vent plug of FIG. 3A coupled to an example housing, illustrating the vent plug in the first position, according to some embodiments;

FIG. 3C is a cross-sectional view of the vent plug of FIG. 3A coupled to the housing, illustrating the vent plug of FIG. 3A in the first position, according to some embodiments;

FIG. 3D is a cross-sectional view of the vent plug of FIG. 3A coupled to the housing, illustrating the vent plug of FIG. 3A in the second position, according to some embodiments;

FIG. 3E is a cross-sectional view of a proximal end of the catheter system of FIG. 3A, illustrating the vent plug of FIG. 3A in the first position, according to some embodiments;

FIG. 3F is a cross-sectional view of a proximal end of the catheter system of FIG. 3A, illustrating the vent plug of FIG. 3A in the second position, according to some embodiments;

FIG. 3G is a cross-sectional view of the vent plug of FIG. 3A coupled to the housing, illustrating the vent plug of FIG. 3A in the first position and an example filter membrane disposed at a distal end of an example extension, according to some embodiments;

FIG. 3H is a lower perspective view of the vent plug of FIG. 3A coupled to the

housing, illustrating the vent plug of FIG. 3A in the first position and the filter membrane disposed at the distal end of the extension, according to some embodiments

FIG. 3I is a cross-sectional view of the vent plug of FIG. 3A coupled to the housing and the housing coupled to the adapter, illustrating the vent plug of FIG. 3A in the first position and the filter membrane disposed at the distal end of the extension, according to some embodiments;

FIG. 4A is an upper perspective view of the catheter system having an example vent plug, according to some embodiments;

FIG. 4B is an upper perspective view of the vent plug of FIG. 4A coupled to an example connector, illustrating the vent plug in the second position, according to some embodiments;

FIG. 4C is a lower perspective view of the vent plug of FIG. 4A, according to some embodiments;

FIG. 4D is a cross-sectional view of the connector according to some embodiments;

FIG. 4E is a cross-sectional view of the vent plug of FIG. 4A coupled to the connector, illustrating the vent plug in the first position, according to some embodiments;

FIG. 4F is a cross-sectional view of the vent plug of FIG. 4A coupled to the connector, illustrating the vent plug in the second position, according to some embodiments;

FIG. 5A is a cross-sectional view of an example vent plug, according to some embodiments; and

FIG. 5B is a cross-sectional view of the vent plug of FIG. 5A coupled to an example connector, according to some embodiments.

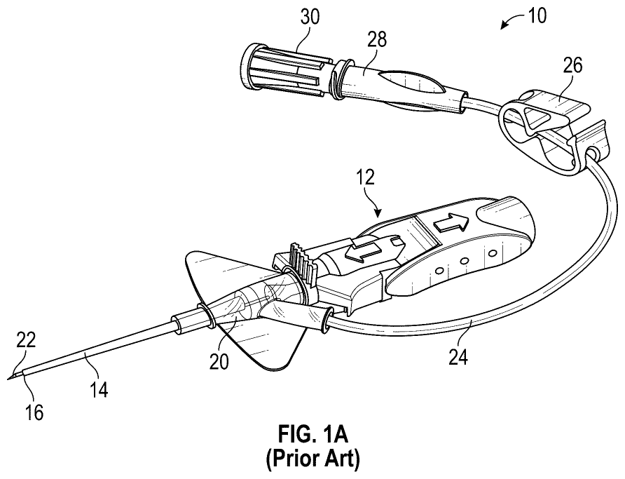

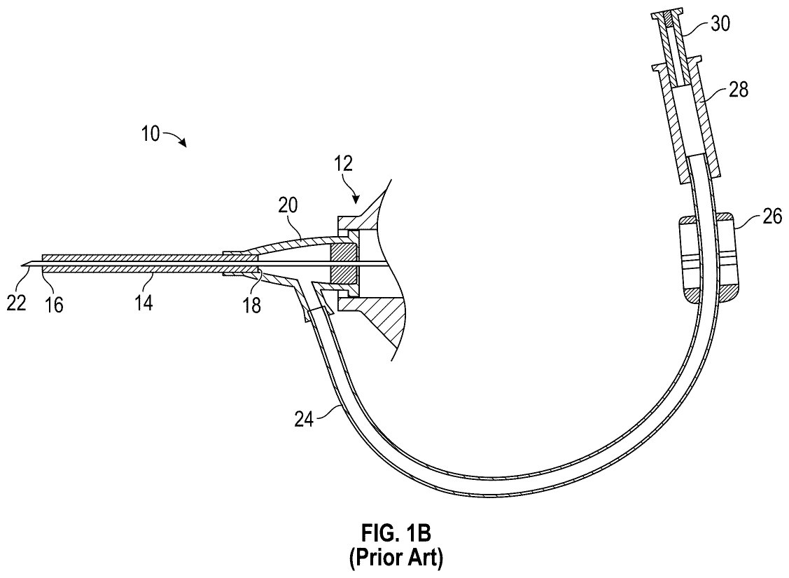

### DESCRIPTION OF EMBODIMENTS

For clarity it is to be understood that the word “proximal” refers to a direction relatively closer to a clinician using the catheter system described in the present disclosure, and the word “distal” refers to a direction relatively further from the clinician using the catheter system described in the present disclosure. For example, the end of a catheter placed within the body of a patient is considered a distal end of the catheter, while the catheter end remaining outside the body is considered a proximal end of the catheter. Also, the words “including,” “has,” and “having,” as used in the present disclosure, including the claims, shall have the same meaning as the word “comprising.”

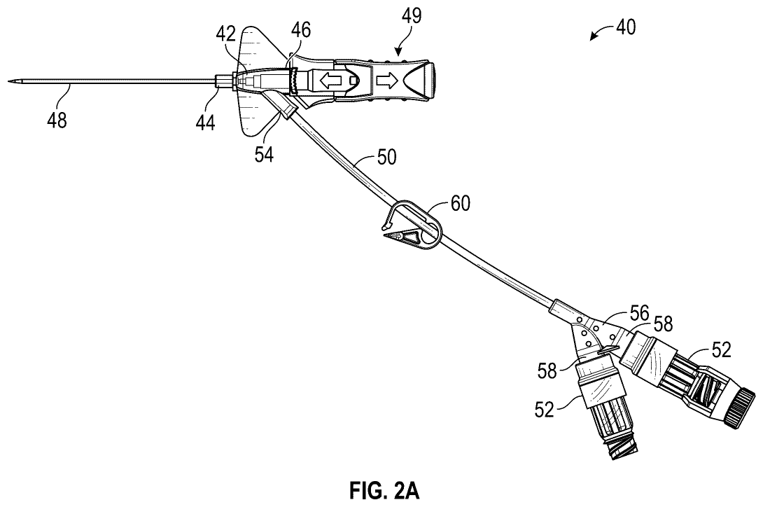

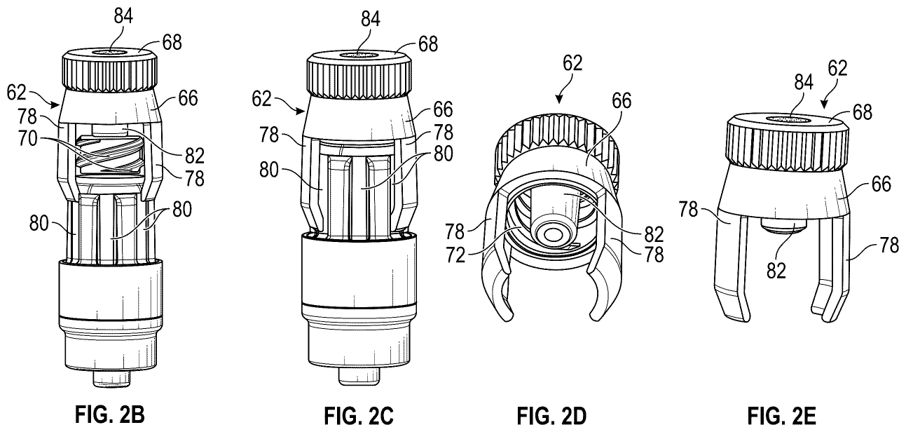

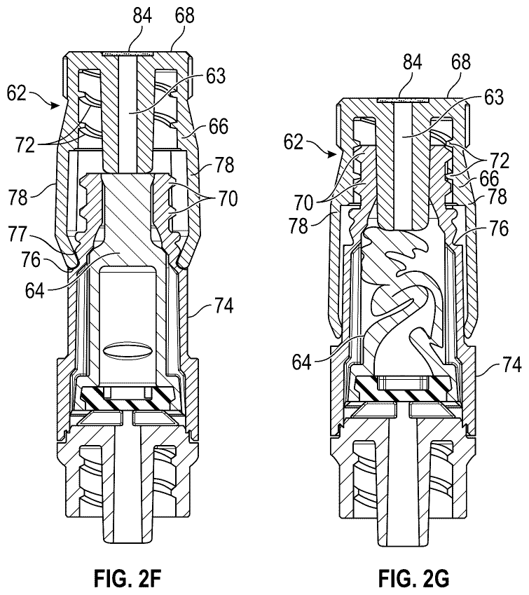

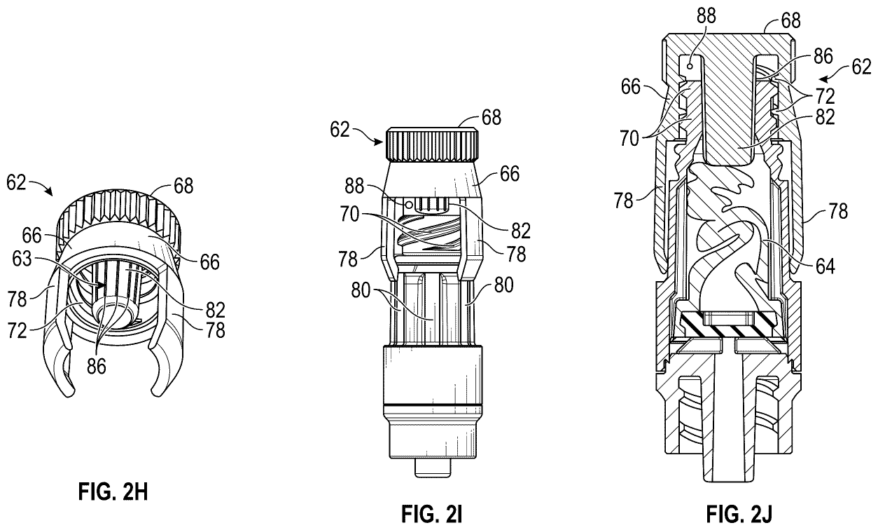

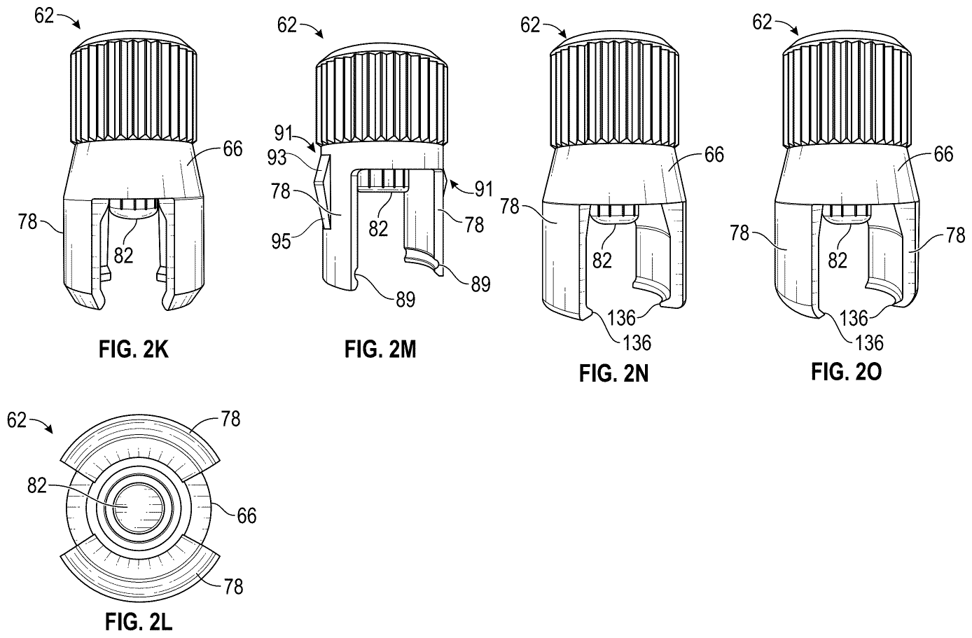

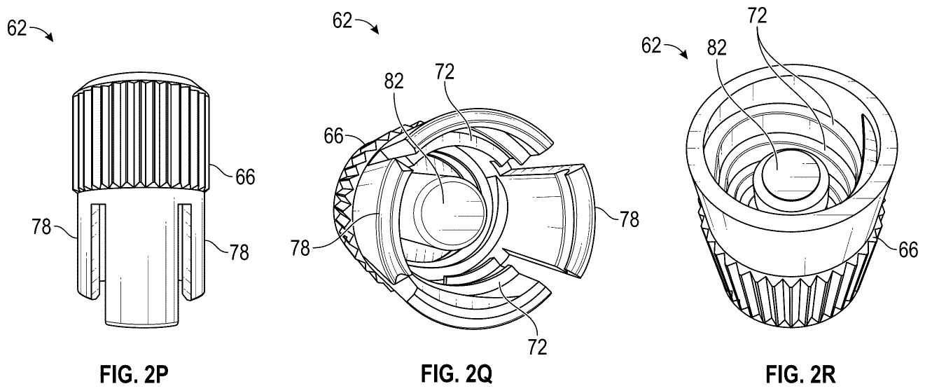

Referring now to FIGS. 2A-2G, in some embodiments, a catheter system 40 may include a catheter adapter 42, which may include a distal end 44 and a proximal end 46. In some embodiments, the catheter system 40 may include a catheter 48 extending distally from the distal end 44 of the catheter adapter 42. In some embodiments, the catheter 48 may be a peripheral intravenous catheter (PIVC), a midline catheter, a peripherally-inserted central catheter, an arterial catheter, or another type of catheter. In some embodiments, a needle assembly 49 may be coupled to the proximal end 46 of the catheter adapter 42, and an introducer needle of the needle assembly 49 may extend through the catheter 48 to aid in introduction of the catheter 48 into vasculature of the patient, including a vein or an artery.

In some embodiments, the catheter system 40 may include an extension tube 50 in fluid communication with the catheter adapter 42 and the catheter 48. In some embodiments, the catheter system 40 may include a connector 52 proximal to the extension tube 50 and in fluid communication with the extension tube 50. In some embodiments, the catheter adapter 42 may include a side port 54 disposed between the distal end 44 of the catheter adapter 42 and the proximal end 46 of the catheter adapter 42. In some embodiments, the extension tube 50 may include a distal end integrated with the side port 54.

In some embodiments, the extension tube 50 may include a proximal end integrated with an adapter 56 having one or more ports 58. In some embodiments, the connector 52 may be coupled to one of the ports 58 of the adapter 56. In some embodiments, the extension tube 50 may be flexible and provide an increased distance between the adapter 56 and the catheter adapter 42 to reduce a risk of disturbing the catheter 48 within the vasculature when an IV fluid supply, a blood collection device, or other device is coupled to the catheter system 40. In some embodiments, a clamp 60 may be disposed on the extension tube 24.

In some embodiments, the catheter system 40 may include a vent plug 62 movable from a first position and movable to a second position. In some embodiments, the vent plug 62 may include a venting pathway 63 that is permeable to air and not blood and that allows air to flow from inside the catheter system 40 to the ambient environment. In some embodiments, when the vent plug is in the first position, a septum 64 (see FIGS. 2F-2G and 2J) of the connector 52 may seal a body 66 of the connector 52 such that the catheter system 40 is a closed catheter system, thereby preventing blood from the patient from escaping from the catheter system 40 to the ambient environment.

In some embodiments, in response to movement of the vent plug 62 from the first position to the second position, the vent plug 62 may be configured to penetrate the septum 64 such that air is vented through the septum 64 and the vent plug 62 to facilitate blood flashback into the extension tube 50. In these embodiments, air may travel from inside the catheter system 40 through the venting pathway 63 and into the ambient environment. In some embodiments, in response to movement of the vent plug 62 from the first position to the second position, blood flashback may travel into the extension tube 50 to provide easy confirmation for the clinician of entry of the catheter 48 into the vasculature of the patient. In some embodiments, when the vent plug 62 is in the second position, the closed catheter system may also be maintained, thereby preventing blood from the patient from escaping from the catheter system 40 to the ambient environment.

In some embodiments, the connector 52 may include a needleless access connector. In some embodiments, the needleless access connector may include a MAXZERO™ needleless connector available from Becton, Dickinson & Company of Franklin Lakes, New Jersey, or another suitable needleless connector. In some embodiments, the connector 52 may correspond to the needleless connector described further in U.S. Pat. No. 10,653,879, filed Jul. 3, 2018, entitled NEEDLELESS CONNECTOR WITH COMPRESSIBLE VALVE, which is hereby incorporated by reference in its entirety. Needleless access connectors generally have a septum inside that self-seals after each use. When the clinician connects a device, such as the IV fluid supply or the blood collection device, to the needleless access connector, the device may push against the septum, temporarily displacing the septum to allow fluid or medication to flow through the catheter system 40. Once the device is disconnected, the septum of the needleless access connector reseals automatically, maintaining the closed, sterile environment.

In some embodiments, a particular needleless access connector may be pre-attached to each of the ports 58 to provide the closed catheter system, reducing a risk of blood stream infections. This may also allow the vent plug 62 to be coupled to the connector 52 and the device (such as the IV fluid supply or the blood collection device) to be coupled to another needleless access connector without removal of the vent plug 62. In some embodiments, the vent plug 62 may be pre-attached to the connector 52 in the first position to reduce a risk of contamination of the catheter system 40 and thereby reduce the risk of blood stream infections.

In some embodiments, the body 66 of the connector 52 may include a proximal end 68 having exterior threading 70. In some embodiments, the vent plug 62 may include interior threading 72, as illustrated, for example, in FIG. 2E. In some embodiments, the exterior threading 70 may be spaced apart from the interior threading 72 when the vent plug 62 is in the first position, as illustrated, for example, in FIG. 2B. In some embodiments, the vent plug 62 may be configured to be pushed distally from the first position until the interior threading 72 contacts the exterior threading 70. In some embodiments, after the interior threading 72 contacts the exterior threading 70, the interior threading 72 may be configured to be threaded to the exterior threading 70 to move the vent plug 62 to the second position, as illustrated, for example, in FIG. 2C.

In some embodiments, the body 66 of the connector 52 may include an outer surface 74 having a groove 76, which may be annular. In some embodiments, the outer surface 74 may include a neck 77 or protrusion, which may be proximate the groove 76. In some embodiments, the vent plug 62 may include multiple arms 78 disposed within the groove 76 in the first position to resist proximal movement of the vent plug 62 with respect to the connector. In some embodiments, the vent plug 62 may include two or three or four arms, for example, to stabilize the vent plug 62 with respect to the connector 52. In some embodiments, the connector 52 may be multiple pieces attached to each other or may be monolithically formed as a single unit.

In some embodiments, the outer surface 74 may include multiple grooves 80. In some embodiments, the arms 78 may be disposed within the grooves 80. In some embodiments, in response to movement of the vent plug 62 from the first position to the second position, the arms 78 may be configured to slide distally within the grooves 80, and the grooves 80 act as guides for the arms 78.

In some embodiments, the vent plug 62 may include an extension 82 configured to penetrate the septum 64 in response to movement of the vent plug 62 from the first position to the second position. In some embodiments, the extension 82 may include a tapered male luer or cannula. In some embodiments, the septum 64 may be constructed of silicone, an elastomer, or another flexible material to facilitate compression and/or resilience of the septum 64.

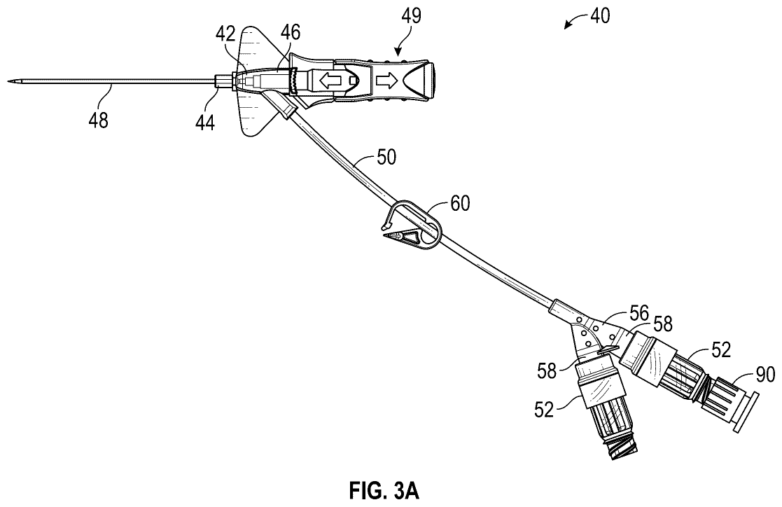

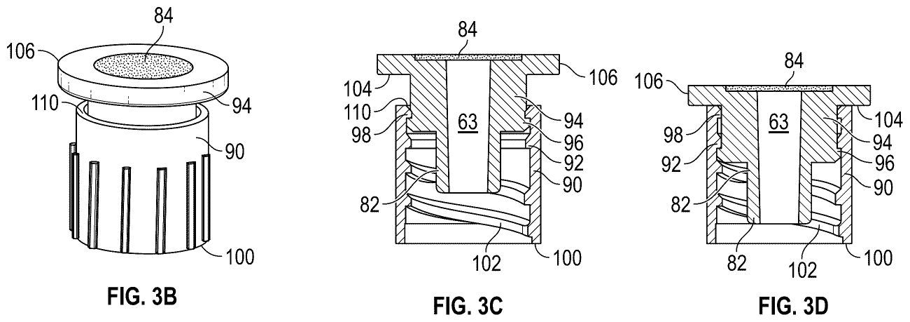

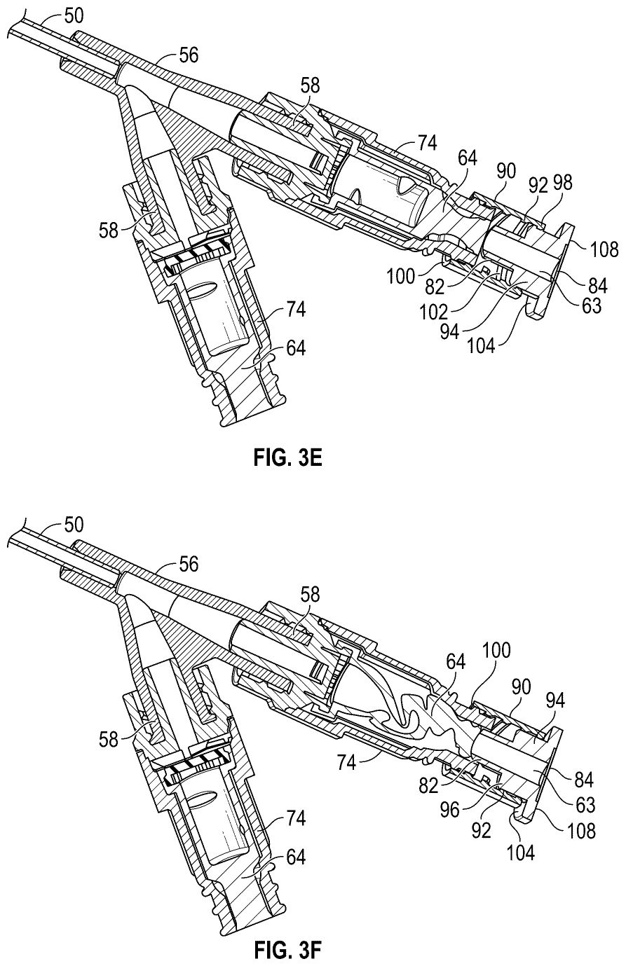

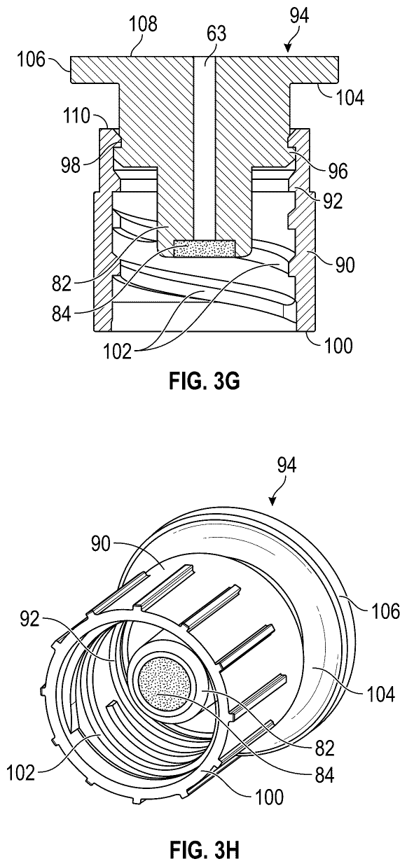

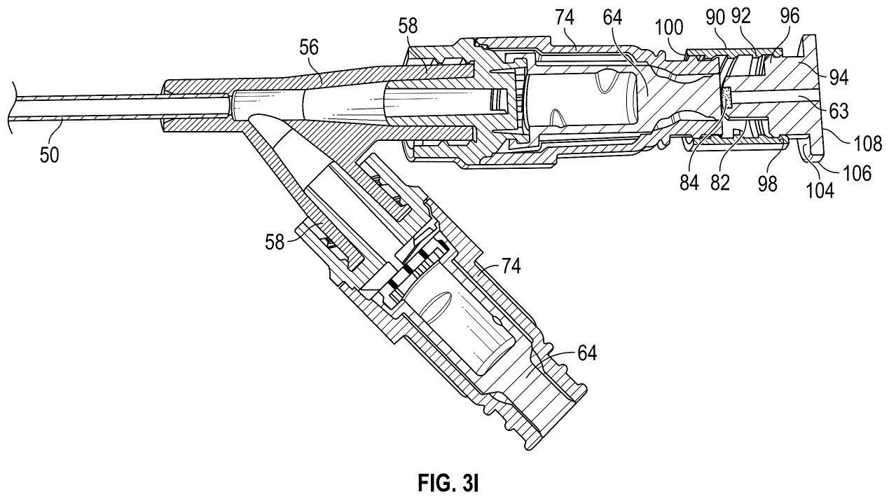

In some embodiments, the venting pathway 63 may extend through an interior of the extension 82, as illustrated, for example, in FIGS. 2F-2G. In these embodiments, the venting pathway 63 may include a central lumen within the extension 82 and extending through the proximal end 68. In some embodiments, the vent plug 62 may include a filter membrane 84 disposed at a proximal end of the venting pathway 63. In some embodiments, the filter membrane 84 may be disposed at a distal end of the venting pathway 63, as illustrated, for example, in FIG. 3G. In some embodiments, the filter membrane 84 disposed at the distal end of the venting pathway 63 may ensure the filter membrane 84 is not damaged by external matter. In some embodiments, the filter membrane 84 may include pores sized to enable flow of air therethrough but prevent flow of blood or other liquids therethrough. For example, the pores could be 0.2 microns or another suitable size.

Referring now to FIGS. 2H-2J, the venting pathway 63 may include one or more grooves 86 disposed on an exterior of the extension 82. In these embodiments, the vent plug 62 may not include the filter membrane 84. In some embodiments, the grooves 86 may be sized to be permeable to air but not blood. In some embodiments, when the vent plug 62 is in the second position, the grooves 86 may allow air to pass through the connector 52. In some embodiments, the vent plug 62 may include a hole 88 that air may pass through to the ambient environment after travelling proximally through the grooves 86. In some embodiments, the hole 88 may be disposed in a collar of the vent plug 62. In some embodiments, the hole 88 may be sized to pass air and not blood as a second line of defense against blood leakage, in addition to the grooves 86.

In some embodiments, a method of observing blood flashback to confirm placement of the catheter 48 within a blood vessel may include taking the catheter system 40 from a package and ensuring the vent plug 62 is attached and the clamp 60 is open or unclamped. Next, in some embodiments, the method may include removing a needle cover and pulling and pushing the vent plug 62 slightly to remove initial tip adhesion between the extension 82 and the septum 64. In some embodiments, the method may then include inserting the catheter system 40 into the patient.

In some embodiments, when the vent plug 62 is in the first position, the septum 64 may seal the body 66 such that the catheter system 40 is a closed catheter system. In some embodiments, after inserting the catheter system 40 into the patient, the method may include moving the vent plug 62 from the first position to the second position to start venting and allow flashback into the extension tube 50. In some embodiments, the vent plug 62 may be moved from the first position to the second position when the clamp 60 is open or unclamped. In some embodiments, in response to movement of the vent plug 62 from the first position to the second position, the vent plug 62 may penetrate the septum 64 such that air is vented through the septum 64 and the vent plug 62, and blood flashback is observed in the extension tube 50. In some embodiments, blood flashback within the extension tube 50 may provide an easy-to-see visible cue in order for the clinician to confirm the catheter 48 is in the vasculature of the patient. In some embodiments, the catheter 48 may be advanced into the vasculature and/or a needle safety mechanism activated after blood flashback is observed.

FIGS. 2K-2Q illustrate further example variations of the vent plug 62 of FIG. 2. As illustrated, for example, in FIGS. 2K-2L, the vent plug 62 may include two of the arms 78, which may each extend about 120° around a circumference of the vent plug 62 and may be spaced apart by opposing gaps. In these embodiments, the arms 78 may provide stability and an increased contact area with a portion of a particular connector when the vent plug 62 moves from the first position to the second position. As illustrated, for example, in FIG. 2M, a distal end of each of the arms 78 may include a groove 89. In some embodiments, the groove 89 may facilitate securement of the arms 78 to the neck 77 or other protrusion of the connector 52 when the vent plug 62 is in the first position (see also FIG. 2F).

In some embodiments, an outer surface of the arms 78 may each include a support rib 91, which may enhance the strength of the arms 78. The support rib 91 is illustrated, for example, in FIG. 2M. In some embodiments, the support rib 91 may include a proximal surface 93 angled with respect to a distal surface 95 to form a generally triangular shape, which may to facilitate gripping by the clinician when the clinician removes the vent plug 62, for example.

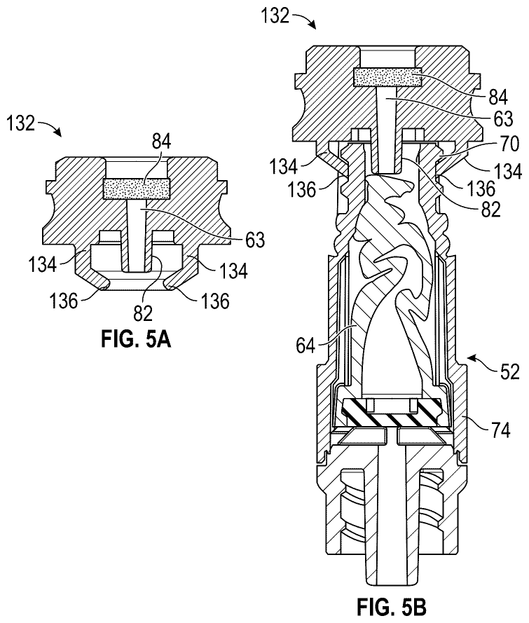

In some embodiments, the distal end of each of the arms 78 may include inwardly extending protrusions 136 similar to FIGS. 5A-5B and configured to snap onto a portion of the connector 52 or another suitable connector. In some embodiments, the inwardly extending protrusions 136 may be rounded, as illustrated in FIG. 2N, or pointed, as illustrated in FIG. 2O. In these embodiments, a shape of the distal end of the arms 78 may be selected based on, for example, a shape of a corresponding feature of a particular connector configured to couple with the inwardly extending protrusions 136 and/or a desired tightness of fit between the particular connector and the vent plug 62 when the vent plug 62 is in the first position.

As illustrated, for example, in FIGS. 2O-2P, in some embodiments, the interior threading 72 may be disposed on other arms of the vent plug 62 that may be spaced apart from the arms 78 by longitudinal gaps aligned with a central axis of the vent plug 62.

As illustrated, for example, in FIG. 2Q, in some embodiments, instead of multiple of the arms 78, a distal end of the vent plug 62 may include an annular ring. In some embodiments, the interior threading 72 may be spaced apart and proximal to a distal end of the annular ring such that the vent plug 62 is configured to be pushed distally from the first position until the interior threading 72 contacts the exterior threading 70. In some embodiments, the interior threading may then be threaded to the exterior threading 70 to reach the second position. In some embodiments, the distal end of the annular ring may include the inwardly extending protrusions 136 (not illustrated in FIG. 2Q).

Referring now to FIGS. 3A-3I, in some embodiments, the catheter system 40 may include a housing 90 coupled to the connector 52 and having an interior protrusion 92. In some embodiments, a vent plug 94 may include an exterior protrusion 96. In some embodiments, the vent plug 94 may be similar or identical to the vent plug 62 of FIGS. 2A-2J in terms of one or more features and/or operation. In some embodiments, in response to movement of the vent plug 62 from the first position to the second position, the exterior protrusion 96 may be configured to snap past the interior protrusion 92 to resist return of the vent plug 62 to the first position. In some embodiments, the interior protrusion 92 may be annular, and/or the exterior protrusion 96 may be annular.

In some embodiments, the housing 90 may include another interior protrusion 98 proximal to the interior protrusion 92. In some embodiments, when the vent plug 94 is in the first position, the other interior protrusion 98 may contact a proximal surface of the exterior protrusion 96 to resist proximal movement of the vent plug 94 with respect to the housing 90.

In some embodiments, a distal end 100 of the housing 90 may include threading 102 threaded to the connector 52. In some embodiments, the vent plug 94 may include a stepped surface 104 forming a push tab 106 or flange at a proximal end 108 of the vent plug 94. In some embodiments, the push tab 106 may facilitate gripping of the vent plug 94 by the clinician to move the vent plug 94 from the first position to the second position. In some embodiments, the stepped surface 104 may be perpendicular or general perpendicular to the central axis of the vent plug 94 and the housing 90. In some embodiments, in response to movement of the vent plug 94 from the first position to the second position, the push tab 106 may contact a proximal end 110 of the housing 90. In some embodiments, the vent plug 94 may include the extension 82 configured to penetrate the septum 64 in response to movement of the vent plug 94 from the first position to the second position.

In some embodiments, the venting pathway 63 may extend through an interior of the extension 82. In these embodiments, the venting pathway 63 may include the central lumen within the extension 82 and extending through the proximal end 68. In some embodiments, the vent plug 62 may include the filter membrane 84 disposed at the proximal end of the venting pathway 63, as illustrated, for example, in FIG. 3B-3F. In some embodiments, the filter membrane 84 may be disposed at the distal end of the venting pathway 63, as illustrated, for example, in FIG. 3G-3H.

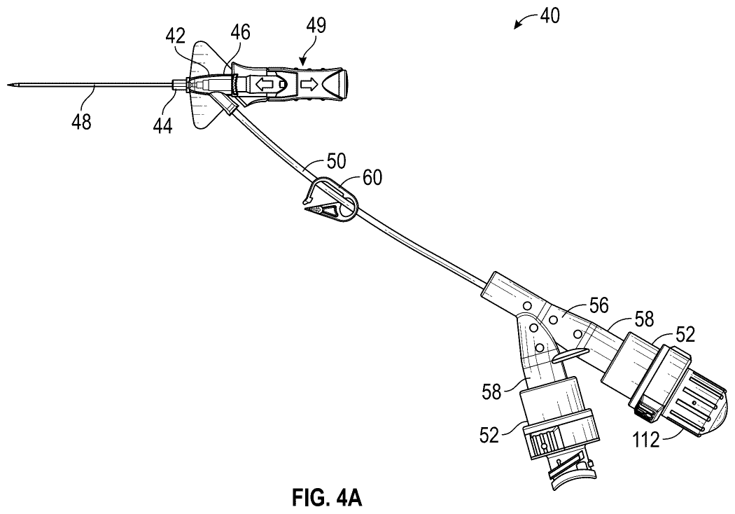

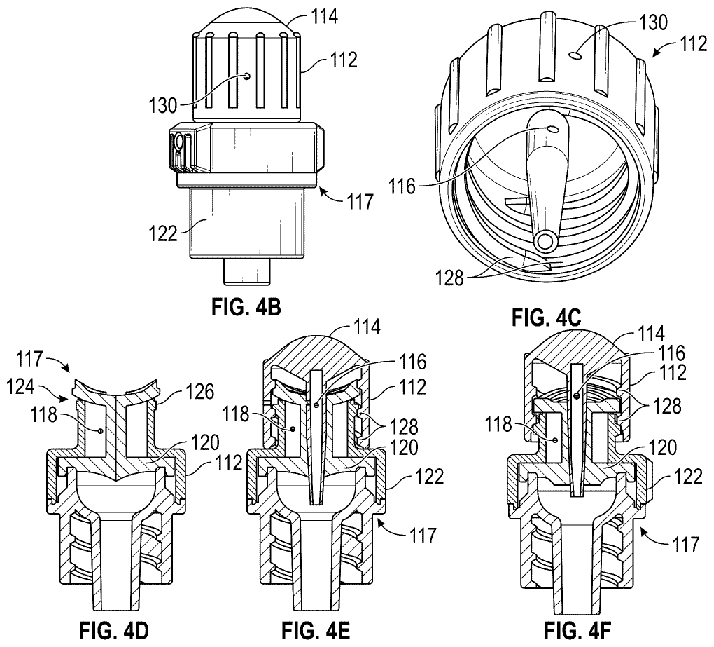

Referring now to FIGS. 4A-4F, a vent plug 112 is illustrated, according to some embodiments. In some embodiments, the vent plug 112 may be similar or identical to the vent plug 62 of FIGS. 2A-2J and/or the vent plug 94 of FIGS. 3A-3I in terms of one or more features and/or operation. In some embodiments, a proximal end 114 of the vent plug 112 may be closed. In some embodiments, the extension 82 may include a first hole 116 permeable to air and not to blood, which may prevent blood leakage from the catheter system 40.

In some embodiments, a connector 117 may be coupled to the vent plug 112. In some embodiments, the connector 117 may be similar or identical to the connector 52 of FIGS. 2 and 3 in terms of one or more features and/or operation. In some embodiments, the connector 117 may include a Q-SYTE™ needle-free connector available from Becton, Dickinson & Company of Franklin Lakes, New Jersey, or another suitable needleless connector. In some embodiments, the connector 117 may be multiple pieces attached to each other or may be monolithically formed as a single unit. In some embodiments, the connector 117 may include a first piece, a second piece, and the septum 120 pinched between the first piece and the second piece. In some embodiments, a distal end of the connector 117 may include a male luer and/or threading surrounding the male luer.

In some embodiments, the vent plug 112 may include a second hole 118, which may be permeable to air and not to blood. as a second line of defense against blood leakage, in addition to the first hole 116. In some embodiments, when the vent plug 112 is in the first position, the first hole 116 may be positioned within a septum 120 that is disposed within a body 122 of the connector 117. In some embodiments, the septum 120 may seal the first hole 116 when the vent plug 112 is in the first position, preventing air from traveling through the first hole 116. In some embodiments, the extension 82 may be elongated and positioned within a slit of the septum 120 to provide the seal of the first hole 116. In some embodiments, in response to movement of the vent plug 112 from the first position to the second position, the first hole 116 may be positioned proximal to the septum 120, and air may be configured to vent through the first hole 116 and the second hole 118.

In some embodiments, the connector 117 may include a proximal end 124 having exterior threading 126, and the vent plug 112 may include a collar 127 having interior threading 128. In some embodiments, the interior threading 128 may be unthreaded with respect to the exterior threading 126 to move the vent plug 112 from the first position to the second position. In some embodiments, the collar 127 may include the second hole 118, and the second hole 118 may be disposed proximal to the septum 120 when the vent plug 112 is in the second position.

Referring now to FIGS. 5A-5B, a vent plug 132 is illustrated, according to some embodiments. In some embodiments, the vent plug 132 may be similar or identical to one or more of the vent plug 62 of FIGS. 2A-2J, the vent plug 94 of FIGS. 3A-3I, and the vent plug 112 of FIGS. 4A-4C and 4E-4F in terms of one or more features and/or operation. In some embodiments, the vent plug 132 may include multiple arms 134 having inwardly extending protrusions 136. In other embodiments, the vent plug 132 may include two or three or four arms, for example, to stabilize the vent plug 132 with respect to the connector 52 or another suitable connector.

In some embodiments, when the vent plug 132 is disposed in the first position, the inwardly extending protrusions 136 snap onto the exterior threading 70 of the connector 52. In some embodiments, the arms 134 may be configured to bias outwardly to remove the vent plug 132 from the connector for disposal.

All examples and conditional language recited herein are intended for pedagogical objects to aid the reader in understanding the invention and the concepts contributed by the inventor to furthering the art and are to be construed as being without limitation to such specifically recited examples and conditions. Although embodiments of the present inventions have been described in detail, it should be understood that the various changes, substitutions, and alterations could be made hereto without departing from the spirit and scope of the invention.

1. A catheter system, comprising:
a catheter adapter, comprising a distal end and a proximal end;
a catheter extending distally from the distal end of the catheter adapter;
an extension tube in fluid communication with the catheter adapter and the catheter;
a connector proximal to the extension tube and in fluid communication with the extension tube, wherein the connector comprises a body and a septum disposed within the body; and
a vent plug movable from a first position to a second position, wherein the vent plug comprises a venting pathway, wherein the venting pathway is permeable to air and not blood, wherein when the vent plug is in the first position, the septum seals the body, wherein in response to movement of the vent plug from the first position to the second position, the vent plug is configured to penetrate the septum such that air is vented through the septum and the vent plug.

2. The catheter system of claim 1, wherein in response to the vent plug being in the second position, blood flashback is configured to travel into the extension tube.

3. The catheter system of claim 2, wherein the catheter adapter further comprises a side port disposed between the distal end of the catheter adapter and the proximal end of the catheter adapter, wherein the extension tube comprises a distal end integrated with the side port, wherein the extension tube comprises a proximal end integrated with an adapter having a port, wherein the connector is coupled to the port of the adapter.

4. The catheter system of claim 1, wherein the connector comprises a proximal end having exterior threading, wherein the vent plug comprises interior threading, wherein the exterior threading is spaced apart from the interior threading when the vent plug is in the first position, wherein the vent plug is configured to be pushed distally from the first position until the interior threading contacts the exterior threading, wherein after the interior threading contacts the exterior threading, the interior threading is configured to be threaded to the exterior threading to move the vent plug to the second position.

5. The catheter system of claim 1, wherein the vent plug comprises an extension configured to penetrate the septum in response to movement of the vent plug from the first position to the second position, wherein the venting pathway extends through an interior of the extension, wherein the vent plug further comprises a filter membrane disposed at a proximal end of the venting pathway.

6. The catheter system of claim 1, wherein the vent plug comprises an extension configured to penetrate the septum in response to movement of the vent plug from the first position to the second position, wherein the venting pathway comprises a plurality of grooves disposed on an exterior of the extension.

7. The catheter system of claim 1, wherein the connector comprises an outer surface having an annular groove, wherein the vent plug comprises a plurality of arms disposed within the annular groove in the first position to resist proximal movement of the vent plug with respect to the connector.

8. The catheter system of claim 1, wherein the connector further comprises an outer surface having a plurality of grooves, wherein the vent plug comprises a plurality of arms disposed within the plurality of grooves, wherein in response to movement of the vent plug from the first position to the second position, the plurality of arms are configured to slide distally within the plurality of grooves.

9. The catheter system of claim 1, further comprising a housing coupled to the connector and having an interior protrusion, wherein the vent plug comprises an exterior protrusion, wherein in response to movement of the vent plug from the first position to the second position, the exterior protrusion is configured to snap past the interior protrusion to resist return of the vent plug to the first position.

10. The catheter system of claim 9, wherein the interior protrusion is annular, wherein the exterior protrusion is annular.

11. The catheter system of claim 9, wherein the housing comprises another interior protrusion proximal to the interior protrusion, wherein when the vent plug is in the first position, the other interior protrusion contacts a proximal surface of the exterior protrusion to resist proximal movement of the vent plug with respect to the housing.

12. The catheter system of claim 9, wherein a distal end of the housing comprises threading threaded to the connector.

13. The catheter system of claim 9, wherein the vent plug comprises a stepped surface forming a push tab at a proximal end of the vent plug, wherein in response to movement of the vent plug from the first position to the second position, the push tab contacts a proximal end of the housing.

14. The catheter system of claim 9, wherein the vent plug comprises an extension configured to penetrate the septum in response to movement of the vent plug from the first position to the second position, wherein the venting pathway extends through an interior of the extension, wherein the vent plug further comprises a filter membrane disposed at a proximal end of the venting pathway.

15. The catheter system of claim 9, wherein the vent plug comprises an extension configured to penetrate the septum in response to movement of the vent plug from the first position to the second position, wherein the venting pathway extends through an interior of the extension, wherein the vent plug further comprises a filter membrane disposed at a distal end of the venting pathway.

16. The catheter system of claim 1, wherein the vent plug comprises an extension configured to penetrate the septum in response to movement of the vent plug from the first position to the second position, wherein the vent plug comprises a closed proximal end, wherein the extension comprises a first hole permeable to air and not to blood, wherein the vent plug comprises a second hole permeable to air and not to blood, wherein when the vent plug is in the first position, the first hole is disposed within the septum, wherein in response to movement of the vent plug from the first position to the second position, the first hole is positioned proximal to the septum and air is configured to vent through the first hole and the second hole.

17. The catheter system of claim 16, wherein the connector comprises a proximal end having exterior threading, wherein the vent plug comprises a collar having interior threading, wherein the interior threading is unthreaded with respect to the exterior threading to move the vent plug from the first position to the second position.

18. The catheter system of claim 17, wherein the collar comprises the second hole.

19. The catheter system of claim 1, wherein the connector comprises a proximal end having exterior threading, wherein the vent plug comprises a plurality of arms having inwardly extending protrusions, wherein when the vent plug is disposed in the first position, the inwardly extending protrusions snap onto the exterior threading of the connector, wherein the plurality of arms are configured to bias outwardly to remove the vent plug from the connector for disposal.

20. A method of observing blood flashback to confirm placement of a catheter within a blood
vessel, the method comprising:
inserting a catheter system into a patient, wherein the catheter system comprises:
a catheter adapter, comprising a distal end and a proximal end;
a catheter extending distally from the distal end of the catheter adapter;
an extension tube in fluid communication with the catheter adapter and the catheter;
a connector proximal to the extension tube and in fluid communication with the extension tube, wherein the connector comprises a body and a septum disposed within the body; and
a vent plug movable from a first position to a second position, wherein when the catheter system is inserted into the blood vessel, the vent plug is in the first position, wherein the vent plug comprises a venting pathway, wherein the venting pathway is permeable to air and not blood, wherein when the vent plug is in the first position, the septum seals the body such that the catheter system is a closed catheter system,
after inserting the catheter system into the patient, moving the vent plug from the first position to the second position, wherein in response to movement of the vent plug from the first position to the second position, the vent plug penetrates the septum such that air is vented through the septum and the vent plug and the blood flashback is observed in the extension tube.
